(19) United States
(12) Patent Application Publication
(10) Pub. No.: US 2026/0360504 A1
(43) Pub. Date: Nov. 12, 2026

(54) STAGED CONSTRAINT-BASED PHYSIOLOGICAL RECOMMENDATION SYSTEM

(71) Applicant: Jennifer Smith, Davie, FL (US)

(72) Inventor: Jennifer Smith, Davie, FL (US)

(21) Appl. No.: 19/672,929

(22) Filed: May 11, 2026

Related U.S. Application Data
(60) Provisional application No. 63/804,309, filed on May 12, 2025.

(51) Int. Cl. G16H 20/30; G16H 10/60; G16H 50/30

(52) U.S. Cl. CPC G16H 20/30; G16H 10/60; G16H 50/30

(57) ABSTRACT

The present invention relates to a physiological recommendation system that receives user-reported health signals and categorizes each signal into predefined signal types associated with subsets of physiological systems. For each categorized signal, the system activates a limited subset of candidate physiological systems while excluding incompatible systems from further evaluation. Cross-signal interaction increases confidence levels when multiple signals overlap in activating the same candidate system and decreases levels when signals conflict. The system identifies a primary physiological system when its confidence level exceeds a predetermined threshold across a minimum number of categorized signals. System-level recommendations comprising food-based, herbal, and lifestyle interventions are generated based on the identified primary physiological system.

### CROSS-REFERENCE OF RELATED APPLICATION(S)

This application claims priority to U.S. Provisional Application No. 63/804,309, which was filed on May 12, 2025, the contents of which are incorporated herein in their entirety.

### FIELD OF THE INVENTION

The present invention relates to systems and methods, including computer-implemented health recommendation systems and methods, generally with respect to staged constraint-based physiological recommendation methods and systems that progressively narrow candidate physiological systems through sequential signal analysis and cross-signal interaction to generate system-level interventions.

### BACKGROUND OF THE INVENTION

Modern individuals often suffer from chronic health and/or medical symptoms and general discomfort while lacking clear guidance on how their food, herbal and lifestyle choices may support healing and/or overall health. Accordingly, health recommendation systems have been developed to assist users in identifying potential physiological imbalances and generating intervention recommendations. Conventional approaches in this field typically operate by evaluating user-reported symptoms, lifestyle factors, and optional laboratory biomarkers against databases of known health conditions or nutritional deficiencies. However, existing systems are either overly generic or clinically rigid and lack the personalization required to be effective for a specific user. Existing food recommendation systems also lack regional context, often making them ineffective for users of a specific region, and such systems also typically have an incomplete understanding of food as nourishment and a healing and health aid.

Some existing systems employ simultaneous evaluation architectures, wherein all user inputs are processed concurrently against all possible physiological systems or health conditions. Such approaches may generate ranked lists of potential conditions or dietary recommendations based on aggregate scoring algorithms. While these systems can process multiple inputs, they often face computational inefficiency challenges when the number of input variables and candidate systems grow large, as the evaluation space expands exponentially. Additionally, simultaneous evaluation approaches may produce recommendations that address individual symptoms rather than identifying underlying systemic issues, potentially resulting in fragmented or conflicting intervention suggestions.

Other approaches in the field have focused on lifestyle monitoring systems that rely on continuous real-time biosensor data. These systems typically utilize wearable devices or implantable sensors to collect physiological measurements such as heart rate, blood glucose levels, activity patterns, and sleep metrics. The collected data is then processed through pattern-matching algorithms or statistical analysis engines to generate wellness scores or intervention alerts. However, such biosensor-dependent architectures present significant accessibility limitations, as they require users to possess and maintain specialized monitoring equipment. The cost and technical complexity associated with continuous biosensor monitoring may restrict the applicability of these systems to users who have access to such devices and are willing to engage in ongoing data collection protocols.

Conventional symptom-checker systems have also been developed using flat decision tree structures or machine learning classifiers trained on symptom-to-diagnosis mappings. These systems typically prompt users to answer a series of predetermined questions and apply rule-based logic or probabilistic models to arrive at potential diagnoses or health recommendations. However, such approaches may lack the ability to dynamically narrow the evaluation space based on sequential input analysis. Furthermore, many existing systems fail to incorporate cross-signal interaction logic that would allow overlapping inputs to reinforce certain physiological system candidates while conflicting inputs reduce the likelihood of others. This limitation can result in reduced diagnostic precision, as the systems may not effectively distinguish between symptoms that point to a common underlying system versus symptoms that suggest multiple independent issues.

Additionally, existing health recommendation systems often operate at the symptom level rather than the system level. That is, recommendations generated by such systems may be tied directly to individual reported symptoms (e.g., recommending caffeine for fatigue) rather than targeting the underlying physiological system responsible for multiple related symptoms (e.g., addressing iron metabolism dysfunction that manifests as fatigue, cognitive impairment, and temperature regulation issues). This symptom-focused approach may result in incomplete or suboptimal intervention strategies that do not address root causes.

Accordingly, there is a need for improved methods and systems that can efficiently process multiple user-reported health signals, progressively narrow the set of candidate physiological systems through structured constraint-based logic, incorporate cross-signal interaction mechanisms to improve diagnostic precision, and/or generate comprehensive system-level recommendations, preferably without requiring continuous biosensor infrastructure. Such approaches would address the computational inefficiency, diagnostic imprecision, accessibility limitations, and symptom-level reasoning deficiencies observed in existing methods and systems.

### SUMMARY OF THE INVENTION

The present invention relates to systems and methods, including computer-implemented health recommendation systems and methods, that are able to receive user inputs representing physiological signals, process those inputs through a multi-stage constraint-based narrowing model, identify candidate physiological systems, and generate health recommendations. The present invention is preferably able to activate a limited subset of candidate physiological systems while excluding incompatible systems from further evaluation. Cross-signal interaction increases the confidence in a particular candidate physiological system when multiple signals overlap in activating the same candidate system and decreases confidence when signals conflict. The system identifies a primary physiological system when confidence exceeds a predetermined threshold across a minimum number of categorized signals. System-level recommendations comprising food-based, herbal and lifestyle interventions are generated based on the identified primary physiological systems.

### BRIEF DESCRIPTION OF THE DRAWINGS

FIG. 1 illustrates a physiological recommendation system according to an exemplary embodiment of the present invention.

FIG. 2 illustrates a signal classification module according to an exemplary embodiment of the present invention.

FIG. 3 illustrates a cross-signal interaction module according to an exemplary embodiment of the present invention.

FIG. 4 illustrates a threshold-based selection module according to an exemplary embodiment of the present invention.

FIG. 5 illustrates a user interface module according to an exemplary embodiment of the present invention.

FIG. 6 illustrates a case study according to an exemplary embodiment of the present invention.

FIG. 7 illustrates a staged constraint-based physiological recommendation system according to an exemplary embodiment of the present invention.

FIG. 8 illustrates a staged constraint-based physiological recommendation system according to an exemplary embodiment of the present invention.

FIG. 9 illustrates a longitudinal system mapping interface showing temporal progression of user inputs, system relationships, and identified patterns according to an exemplary embodiment of the present invention.

FIG. 10 illustrates an output interface of the system according to an exemplary embodiment of the present invention.

FIG. 11 illustrates system workflow incorporating geolocation-based recommendation functionality according to an exemplary embodiment of the present invention.

FIG. 12 illustrates a general system architecture according to an exemplary embodiment of the present invention.

### DESCRIPTION OF THE INVENTION

The present invention is described herein in exemplary form. The description herein is not intended to limit the scope of the invention or this patent. Rather, it is contemplated that the claimed subject matter might also be embodied in other ways, to include additional features or combinations of features similar to the ones described herein.

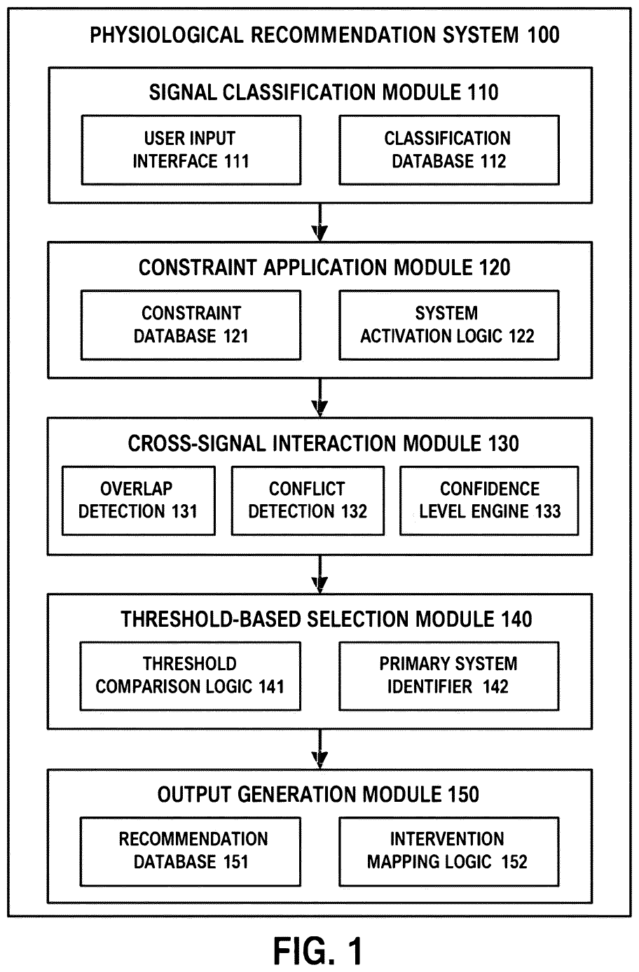

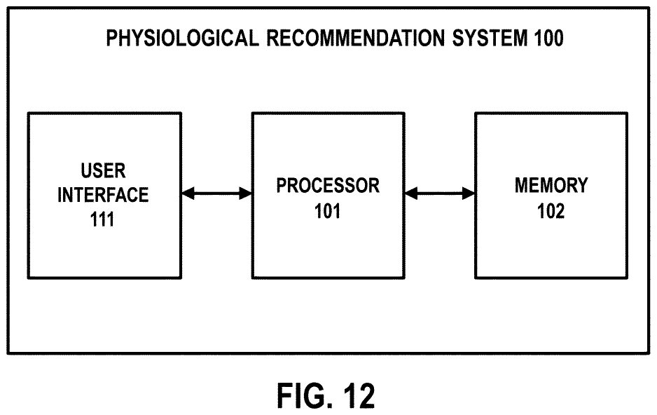

In an exemplary embodiment, the present disclosure relates to a staged constraint-based physiological recommendation system 100 that progressively narrows candidate physiological systems through sequential signal analysis and cross-signal interaction to generate system-level interventions, preferably without requiring real-time biosensor data. Those of ordinary skill in the art will recognize that the physiological recommendation system 100 may be preferably implemented via a processor 101 and a memory 102 storing instructions that, when executed, implement for example functional modules operating in a staged reduction process, for example signal classification 110, constraint application 120, cross-signal interaction 130, threshold-based selection 140, and output generation 150, as illustrated in exemplary form in FIGS. 1 and 12.

As used herein, the term “user-reported health signal” or “health signal” refers to any subjective symptom, lifestyle factor, dietary habit, or laboratory biomarker value provided through an interface, preferably a user interface 111, wherein such signals preferably do not require real-time biosensor monitoring and may include but are not limited to qualitative descriptions (e.g., fatigue, brain fog, cold sensitivity) and quantitative measurements (e.g., ferritin level of 21 ng/mL, heavy menstrual cycles lasting 7 days).

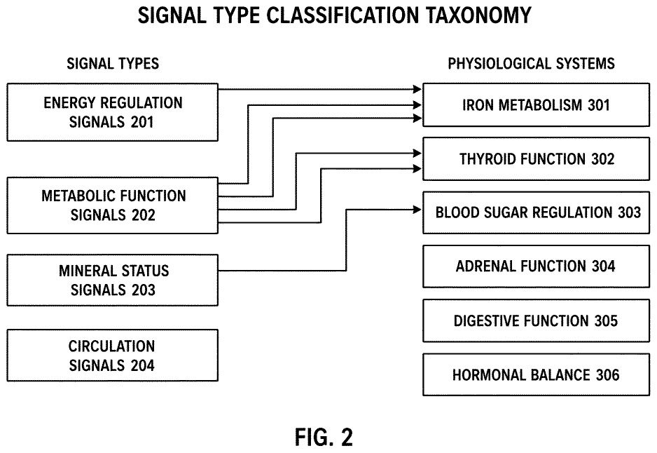

As used herein, the term “signal type” refers to a predefined category into which user-reported health signals and/or health signals are classified, wherein each signal type is associated with a subset of candidate physiological systems. By way of example only, signal types may include but are not limited to energy regulation signals 201, metabolic function signals 202, mineral status signals 203, and circulation signals 204, as is illustrated in exemplary form in FIG. 2. By way of example only, each signal type may be associated with at least one candidate physiological system, for example associated with 2 to 8 candidate physiological systems, from a plurality of physiological systems. By way of example only, and without limitation, candidate physiological system may include but are not limited to iron metabolism 301, thyroid function 302, blood sugar regulation 303, adrenal function 304, digestive function 305 and hormonal balance systems 306, as is illustrated in exemplary form in FIG. 2. Those of ordinary skill in the art will recognize that there are other candidate physiological systems that are within the scope of the present invention that are not mentioned specifically herein.

As used herein, the term “limited subset” means by way of example at least one candidate physiological system, preferably 2 to 8 candidate physiological systems, activated by a single signal type. By way of example, those of ordinary skill in the art will recognize that by a “limited subset” the present invention need not evaluate all physiological systems simultaneously. By way of example, such a constraint-based approach of the present invention is able to progressively eliminate incompatible systems at each stage of signal processing.

As used herein, the term “overlap” means that two or more categorized signals activate the same candidate physiological system, thereby increasing the confidence level for that system. The term “conflict” means categorized signals provide contradictory indications (e.g., a high energy signal conflicts with a fatigue signal, or normal thyroid laboratory values conflict with hypothyroid symptoms), thereby decreasing the confidence levels for affected systems.

As used herein, the term “system-level” means recommendations targeting the underlying physiological system rather than individual symptoms. By way of example, system-level recommendations for iron metabolism may include iron-rich foods, herbal support such as nettle and supporting nutrients such as vitamin C. In contrast, by way of example, symptom-level recommendations would address individual symptoms such as recommending caffeine for fatigue without addressing the underlying iron deficiency.

### System Architecture Overview

By way of example, with reference to FIGS. 1 and 12, those of ordinary skill in the art will recognize that the physiological recommendation system 100 comprises a processor 101 and a memory 102 storing instructions that, when executed by the processor 101, cause the system 100 to implement functional modules, preferably a signal classification module 110, a constraint application module 120, a cross-signal interaction module 130, a threshold-based selection module 140 and an output generation module 150. Those of ordinary skill in the art will recognize that the system 100 preferably operates without requiring real-time biosensor data, thereby enabling broader accessibility compared to systems that rely on continuous biosensor monitoring.

Those of ordinary skill in the art will recognize that the processor 101 may be any suitable processing unit capable of executing instructions stored in memory 102, including but not limited to a central processing unit (CPU), a graphics processing unit (GPU), a field-programmable gate array (FPGA), an application-specific integrated circuit (ASIC), or a combination thereof. The memory 102 may comprise volatile memory (e.g., RAM) and non-volatile memory (e.g., ROM, flash memory, hard disk drive, solid-state drive) storing program instructions and data structures including but not limited to a classification database 112, a constraint database 121, and a recommendation database 151.

### Signal Intake and Classification

By way of example, the signal classification module 110 may be configured to receive at least one, preferably a plurality of, user-reported health signals and categorize each signal into one of a plurality of predefined signal types. As illustrated by way of example in FIG. 2, the signal classification module 110 may comprise a signal input interface 111 and a classification database 112. In one exemplary embodiment, the signal input interface 111 receives user-reported health signals through a user interface 111, which may include by way of example text input fields, dropdown menus, slider controls, or voice input interfaces.

By way of example, the user-reported health signals and/or health signals may comprise at least one of subjective symptoms (e.g., fatigue, brain fog, cold sensitivity, headaches, digestive discomfort), lifestyle factors (e.g., sleep duration, stress levels, exercise frequency), dietary habits (e.g., iron intake, caffeine consumption, meal timing) and laboratory biomarker values (e.g., ferritin level, hemoglobin level, thyroid hormone levels, blood glucose levels). However, preferably the system 100 of the present invention is configured to operate without laboratory biomarkers, or preferably with laboratory biomarker values that are non-real-time measurements obtained from periodic testing, without requiring continuous biosensor monitoring. This distinguishes the present system from other systems that rely on real-time biosensor data such as continuous glucose monitors, wearable heart rate monitors and/or implantable sensors.

By way of example, with reference to FIGS. 1 and 2, the classification database 112 may store signal-to-type mappings that define associations between specific user-reported health signals and predefined signal types. For example, in one exemplary embodiment, the classification database 112 may map the symptom fatigue to the signal type energy regulation signals, the symptom brain fog to the signal type metabolic function signals, the symptom cold sensitivity to the signal type circulation signals and/or the laboratory value ferritin level of 21 ng/mL to the signal type mineral status signals. By way of example, the classification database 112 may be implemented as a relational database, a hash table, a decision tree, or any suitable data structure that enables efficient retrieval of signal type classifications.

By way of example, with reference to FIGS. 1 and 2, in operation, in one exemplary embodiment, when a user inputs a health signal such as “fatigue” through the signal input interface 111, the signal classification module 110 queries the classification database 112 to retrieve the corresponding signal type energy regulation signals. By way of example, this categorization process is performed for each user-reported health signal, thereby generating a set of categorized signals that serve as input to the constraint application module 120.

### Constraint Application

By way of example, the constraint application module 120 may be configured to, for each categorized signal, activate a limited subset of candidate physiological systems from a plurality of physiological systems and exclude physiological systems outside said subset from further evaluation. As illustrated in FIG. 1, in one exemplary embodiment, the constraint application module 120 comprises a constraint database 121 and system activation logic 122.

By way of example, with reference to FIG. 2, the constraint database 121 may store signal-to-system mappings that define associations between each predefined signal type and a subset of candidate physiological systems. For example, in one exemplary embodiment, the signal type energy regulation signal 201 may be associated with the candidate physiological system iron metabolism 301 only. In another exemplary embodiment, the signal type energy regulation signal 201 may be associated with candidate physiological systems including iron metabolism 301, thyroid function 302, blood sugar regulation 303, and adrenal function 304, but may exclude digestive function 305 and hormonal balance 306 systems. Similarly, in another exemplary embodiment, the signal type metabolic function signals 202 may be associated with iron metabolism 301, thyroid function 302 and blood sugar regulation 303, while the signal type mineral status signals may be associated primarily with iron metabolism 301, and potentially preferably other mineral-related systems. By way of example, each signal type may be associated with at least one, but less than all, physiological systems, thereby preferably ensuring that the constraint application module 120 activates only a limited subset rather than evaluating all physiological systems simultaneously.

With reference to FIG. 2 by way of example, in one exemplary embodiment the plurality of physiological systems stored in the constraint database 121 comprises at least: iron metabolism 301, thyroid function 302, blood sugar regulation 303, adrenal function 304, digestive function 305 and hormonal balance 306 systems. In certain preferred embodiments, the plurality of physiological systems may further include immune function, cardiovascular function, neurological function, respiratory function, musculoskeletal function and dermatological function systems. By way of example, each physiological system is defined by a set of associated symptoms, biomarkers, and intervention strategies stored in the recommendation database 151.

With reference to FIGS. 1 and 2 by way of example, in one exemplary embodiment, the system activation logic 122 retrieves the list of candidate physiological systems associated with each categorized signal from the constraint database 121 and updates an activation status for each system. By way of example only, when the signal classification module 110 categorizes the symptom fatigue as energy regulation signals, the system activation logic 122 queries the constraint database 121 and activates the candidate physiological systems associated with energy regulation signals, such as in one exemplary embodiment, iron metabolism 301, thyroid function 302, blood sugar regulation 303 and adrenal function 304. In such exemplary embodiment, physiological systems outside this subset, such as digestive function 305 and hormonal balance 30 systems, are excluded from further evaluation at this stage, thereby reducing the computational complexity of subsequent processing steps.

With reference to FIGS. 1 and 2 by way of example, in one exemplary embodiment, the constraint application process is repeated for each categorized signal. For example, when the symptom brain fog” is categorized as metabolic function signals, the system activation logic 122 activates the candidate physiological systems associated with metabolic function signals 202, which may include iron metabolism 301, thyroid function 302 and blood sugar regulation 303. In another exemplary embodiment, systems that are activated by multiple categorized signals (e.g., iron metabolism 301 activated by both energy regulation signals 201 and metabolic function signals 202) are flagged for reinforcement in the cross-signal interaction module 130, while systems that are not activated by a subsequently categorized signal after being previously activated are flagged for reduction in confidence level.

### Cross-Signal Interaction

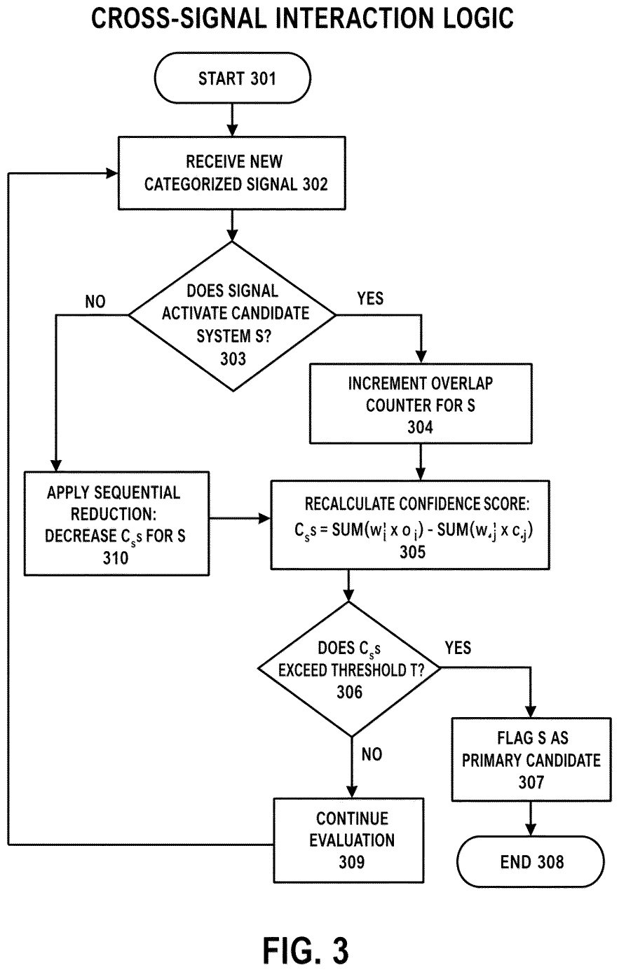

With reference to FIGS. 1 and 2 by way of example, in one exemplary embodiment, the cross-signal interaction module 130 is configured to perform cross-signal interaction by: (i) increasing a confidence level for a candidate physiological system when multiple categorized signals overlap in activating said candidate system, and (ii) decreasing a confidence level for a candidate physiological system when categorized signals conflict. As illustrated in FIGS. 1 and 3, in one exemplary embodiment, the cross-signal interaction module 130 comprises an overlap detection component 131, a conflict detection component 132, and a confidence level engine 133.

With reference to FIGS. 1-3, in one exemplary embodiment, the overlap detection component 131 identifies candidate physiological systems that are activated by multiple categorized signals. For example, in one exemplary embodiment, if the categorized signals energy regulation signals 201 and metabolic function signals 202 both activate iron metabolism 301, the overlap detection component 131 flags iron metabolism as having overlapping signal support. By way of example, in one exemplary embodiment, the degree of overlap may be quantified based on the number of categorized signals that activate the same candidate system, the reliability of each signal type, or a combination thereof.

With reference to FIGS. 1-3, in one exemplary embodiment, the conflict detection component 132 identifies categorized signals that provide contradictory indications. For example, in one exemplary embodiment, if a user reports both high energy and fatigue, these signals conflict and may reduce the confidence level for systems associated with energy regulation. Similarly, in another exemplary embodiment, if a user provides laboratory values indicating normal thyroid hormone levels but reports symptoms consistent with hypothyroidism (e.g., cold sensitivity, fatigue, weight gain), the conflict detection component 132 may flag thyroid function as having conflicting signal evidence and reduces its confidence level accordingly.

In one exemplary embodiment, the confidence level engine 133 determines or calculates a confidence level for each candidate physiological system. In one preferred embodiment, the confidence level is determined or calculated based on the overlap and conflict information provided by the overlap detection component 131 and conflict detection component 132. A confidence level may be determined or calculated for each candidate physiological system.

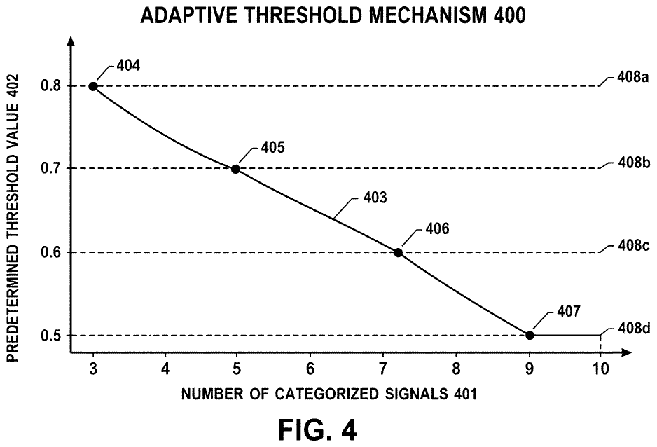

As used herein, the phrase “confidence level” refers generally to an indicator that evaluates or quantifies the degree to which a candidate physiological system is determined to be the underlying physiological system responsible for or associated with the user-reported health signal(s). In one exemplary embodiment, the “confidence level” may evaluate or quantify the degree to which a candidate physiological system is identified as the underlying physiological system responsible for the user-reported health signal(s). In another exemplary embodiment, the “confidence level” may evaluate or quantify the degree to which a candidate physiological system may be identified as a primary physiological system. By way of example only, the confidence level may be compared to or evaluated against a threshold as described in exemplary terms herein, for example as illustrated in FIG. 4. Notably, the threshold need not be fixed but rather the threshold may be dynamic as described herein in exemplary embodiments, for example as illustrated in FIG. 4.

It will be recognized that a number of factors may affect (e.g., decrease or increase) confidence level, for example the confidence level for a candidate physiological system. As one example, overlapping signals that activate the same candidate physiological system may increase confidence level. That is, in one exemplary embodiment, when more than one health signal, preferably two or more categorized signals, activate the same candidate physiological system, the confidence level for that system may be increased.

As another example, conflict reduction from contradictory signals may decrease or increase confidence level. For example, in one exemplary embodiment, signals may provide contradictory indications (e.g., a high energy signal conflicts with a fatigue signal, or normal thyroid laboratory values conflict with hypothyroid symptoms), thereby decreasing the confidence score for the affected systems.

As another example, the reliability of a signal may decrease or increase confidence level. For example, in one exemplary embodiment, one or more signals may be considered more reliable (e.g., more predictive) compared to other signals in identifying a candidate physiological system. Preferably, a more reliable signal is given a greater weigh in the system 100. Those of ordinary skill in the art will recognize that a reliable signal may increase confidence level.

As another example, the severity weighting of a signal may decrease or increase confidence level. For example, in one exemplary embodiment, a signal may be considered more severe (e.g., 102 °F body temperature more severe than 99 °F body temperature), sometimes more severe compared to another signal (e.g., 102 °F body temperature more severe than sneezing), which severity may be preferably expressed as a weighed value. Those of ordinary skill in the art will recognize that a more severe signal may increase confidence level.

As another example, biomarker and biomarker weightings may decrease or increase confidence level. Those of ordinary skill in the art will recognize that biomarkers, for example laboratory biomarker values, typically provide an objective measurement compared to the subjective nature of symptom reports. As such, in one exemplary embodiment, a biomarker may be given a greater weighting in the system 100. Those of ordinary skill in the art will recognize that the use of biomarkers and/or biomarker weightings may increase confidence level.

As another example, the temporal consistency of a signal may decrease or increase confidence level. In one exemplary embodiment, a signal may remain present, continuous and/or stable over a period of time. Those of ordinary skill in the art will recognize that the use of a temporally consistent signal may increase confidence level.

As another example, sequential reinforcement of a signal may decrease or increase confidence level. For example, each new signal strengthens or weakens a previously developing pattern. In one exemplary embodiment, sequential reinforcement refers to a later signal(s) reinforcing a system already supported and/or activated by an earlier signal(s). An exemplary embodiment would be initial signals of brain fog and fatigue activating both iron metabolism and thyroid (so confidence is moderate for both), but once a user adds the new signal ferritin 21, the new signal increases confidence for iron metabolism. Thus, those of ordinary skill in the art will recognize that confidence level changes over time as new signals are introduced in sequence.

In another exemplary embodiment of the present invention, the confidence level calculated by the confidence level engine 133 is a confidence score for each system based on the overlap and conflict information provided by the overlap detection component 131 and conflict detection component 132. In a preferred embodiment, the confidence score is calculated as a weighted sum of signal overlap factors, wherein each signal overlap factor is weighted based on a reliability metric associated with the signal type. For example, in one exemplary embodiment, laboratory biomarker values such as ferritin level may be assigned a higher reliability weight (e.g., 1.0) compared to subjective symptoms such as fatigue (e.g., 0.5), reflecting the objective nature of laboratory measurements versus the subjective nature of symptom reports.

By way of example, in one exemplary embodiment, the confidence scoring engine 133 may implement the following formula to calculate the confidence score (C_s) for a candidate physiological system:

C_s=Σ(w_i×o_i)−Σ(w_j×c_j)

where w_i is the reliability weight for signal type i that activates system s, o_i is an overlap factor (e.g., 1 if signal i activates system s, 0 otherwise), w_j is the reliability weight for signal type j that conflicts with system s, and c_j is a conflict factor (e.g., 1 if signal j conflicts with system s, 0 otherwise). In this exemplary embodiment, this formula enables the confidence level engine 133 to quantify the degree of support for each candidate physiological system based on the combination of overlapping and conflicting signals.

In certain exemplary embodiments, the cross-signal interaction module 130 may further comprise sequential reduction logic that reduces the confidence level for a candidate physiological system when a subsequently categorized signal fails to activate said candidate system after said system was previously activated by an earlier categorized signal. For example, in one exemplary embodiment, if the initial categorized signal energy regulation signals 201 activates thyroid function 302, but a subsequently categorized signal mineral status signals 203 (e.g., ferritin level of 21 ng/mL) does not activate thyroid function 302, the confidence level for thyroid function 302 is reduced. Such sequential reduction logic may enable the system 100 to dynamically adjust system likelihood based on the order and content of received signals, thereby improving diagnostic precision as more information becomes available.

In certain exemplary embodiments, the cross-signal interaction process is performed iteratively as each new health signal is categorized. After the signal classification module 110 categorizes a new signal and the constraint application module 120 activates the corresponding subset of candidate physiological systems, the cross-signal interaction module 130 updates the confidence levels for all candidate systems based on the new signal's overlap and conflict relationships with previously categorized signals. Such an iterative approach may ensure that the confidence levels reflect the cumulative evidence from all user-reported health signals received up to the current point in the evaluation process.

### Threshold-Based Selection

As illustrated in FIGS. 1 and 4, in one exemplary embodiment, the threshold-based selection module 140 may be configured to identify a primary physiological system when said system's confidence level exceeds a predetermined threshold across a minimum number of categorized signals. As illustrated in FIG. 1, by way of example, the threshold-based selection module 140 comprises threshold comparison logic 141 and a primary system identifier 142.

With reference to FIG. 4, by way of example, the predetermined threshold may be a fixed value (e.g., 0.6, 0.7, or 0.8) or may be dynamically adjusted based on a total number of categorized signals received. In a preferred embodiment, the predetermined threshold is dynamically adjusted based on the total number of categorized signals received, wherein the threshold decreases as the number of signals increases. By way of example, this adaptive threshold mechanism balances sensitivity and specificity based on input quantity: when fewer signals are available, a higher threshold is required to ensure sufficient evidence before identifying a primary physiological system; when more signals are available, a lower threshold is acceptable because the cumulative evidence from multiple signals provides greater diagnostic confidence.

With reference to FIG. 4, in one exemplary embodiment, the predetermined threshold may be set to 0.8 when the total number of categorized signals is 3, may decrease to 0.7 when the total number of categorized signals is 4 or 5, may decrease to 0.6 when the total number of categorized signals is 6 or 7, and may decrease to 0.5 when the total number of categorized signals is 8 or more. This adaptive threshold curve, illustrated by way of example in FIG. 4, ensures that the system 100 does not prematurely identify a primary physiological system based on insufficient evidence, while also avoiding overly stringent requirements that would prevent system identification even when substantial evidence is available.

In one exemplary embodiment, the minimum number of categorized signals required for identifying the primary physiological system is preferably at least three signals. Such a requirement seeks to ensure that the system 100 has received sufficient information to distinguish between candidate physiological systems and avoid false positives based on a single symptom or biomarker. In other exemplary embodiments, the minimum number of categorized signals may be set to 4, 5, or more, depending on the desired balance between diagnostic speed and accuracy. In other exemplary embodiments, the minimum number of categorized signals may be set to less than 3 signals.

By way of example, the threshold comparison logic 141 may compare the confidence level of each candidate physiological system to the predetermined threshold. If multiple candidate physiological systems exceed the predetermined threshold, the primary system identifier 142 selects the candidate system with the highest confidence level as the primary physiological system. For example, in one exemplary embodiment, if both iron metabolism 301 (e.g., confidence score 0.85) and thyroid function 302 (e.g., confidence score 0.72) exceed a predetermined threshold of 0.6, the primary system identifier 142 selects iron metabolism 301 as the primary physiological system because it has the highest confidence score.

In certain exemplary embodiments, the system 100 is configured to identify a secondary physiological system when a second candidate system's confidence level exceeds a secondary threshold. By way of example, the secondary threshold may be lower than the primary threshold (e.g., 0.5 compared to 0.6) to accommodate complex cases involving multiple underlying physiological systems. Preferably, when both a primary and secondary physiological system are identified, the output generation module 150 generates supplemental recommendations addressing both systems, thereby providing comprehensive support for users with multiple co-occurring imbalances.

### Output Mapping

With reference to FIG. 1, by way of example, the output generation module 150 is configured to generate a set of recommendations based on the identified primary physiological system. Preferably, said recommendations comprise at least one of: food-based interventions, herbal interventions, and lifestyle interventions. As illustrated in FIG. 1, by way of example, the output generation module 150 comprises a recommendation database 151 and intervention mapping logic 152.

In one exemplary embodiment, the recommendation database 151 stores mappings between each physiological system and corresponding interventions. For example, the recommendation database 151 may map iron metabolism 301 to food-based interventions 616 such as iron-rich foods (e.g., red meat, spinach, lentils, fortified cereals), herbal interventions 617 such as nettle (Urtica dioica) and yellow dock (Rumex crispus), lifestyle interventions such as avoiding tea and coffee consumption with meals (which inhibit iron absorption), and specific nutritional support recommendations 618 such as vitamin C supplementation (which enhances iron absorption) and avoiding calcium supplements concurrent with iron-rich meals.

Similarly, in another exemplary embodiment, the recommendation database 151 may map thyroid function 302 to food-based interventions 616 such as iodine-rich foods (e.g., seaweed, iodized salt, fish), selenium-rich foods (e.g., Brazil nuts, sunflower seeds), herbal interventions 617 such as ashwagandha (Withania somnifera) and bladderwrack (Fucus vesiculosus), lifestyle interventions such as stress reduction techniques and adequate sleep, and specific nutritional support recommendations 618 such as selenium and zinc supplementation. Preferably, each physiological system in the recommendation database 151 is associated with a comprehensive set of interventions targeting that system's underlying function.

By way of example, the intervention mapping logic 152 may retrieve the set of interventions associated with the identified primary physiological system from the recommendation database 151 and generate a structured output comprising at least one, preferably at least two, of: food-based interventions 616, herbal interventions 617, and lifestyle interventions. In preferred embodiments, the set of recommendations further comprises specific nutritional support recommendations 618 comprising at least one of: specific food items, herbal supplements, vitamins, and minerals, selected based on the identified primary physiological system.

In one exemplary embodiment, the recommendations generated by the output generation module 150 are system-level recommendations rather than symptom-level recommendations. For example, the recommendations target the underlying physiological system identified by the threshold-based selection module 140, rather than addressing individual symptoms reported by the user. For example, if the primary physiological system is identified as iron metabolism 301, the recommendations will include interventions that support iron metabolism (e.g., iron-rich foods, nettle, vitamin C) rather than symptom-specific recommendations such as recommending caffeine for fatigue or pain relievers for headaches. This system-level approach provides more comprehensive and effective support by addressing the root cause of multiple related symptoms.

### User Interface and Sequential Prompting

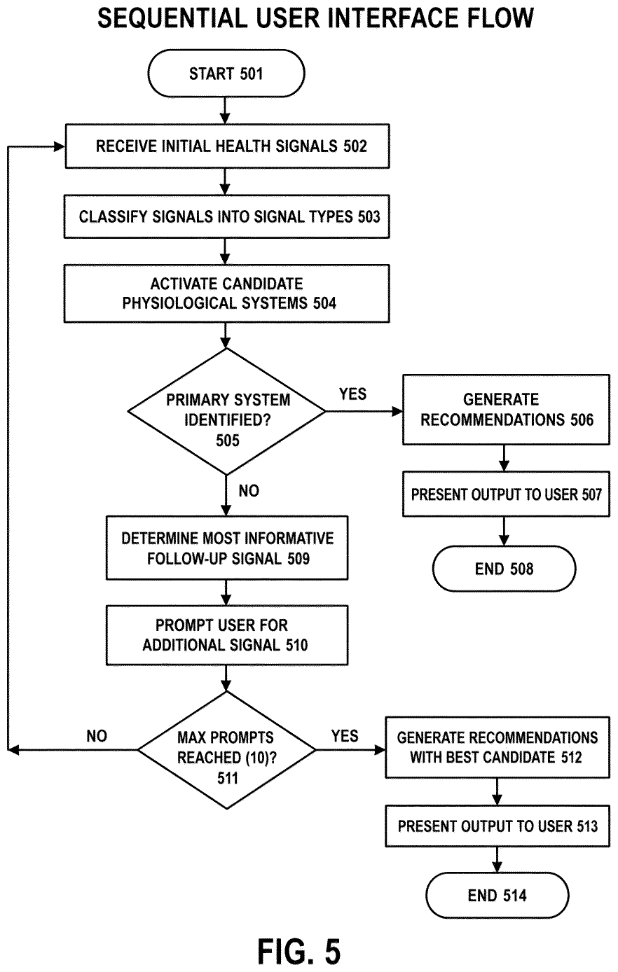

In certain exemplary embodiments, the system 100 further comprises a user interface 111 configured to sequentially prompt the user for additional health signals based on the currently activated subset of candidate physiological systems. As illustrated in FIG. 5, the user interface 111 implements an adaptive questioning mechanism that focuses data collection on the most relevant signals for the activated systems.

In one exemplary embodiment, the user interface 111 operates as follows: after the signal classification module 110 categorizes an initial set of user-reported health signals and the constraint application module 120 activates a subset of candidate physiological systems, the user interface determines which additional signals would be most informative for distinguishing between the currently activated candidate systems. For example, if the currently activated candidate systems include both iron metabolism 301 and thyroid function 302, the user interface 111 may prompt the user for additional signals that are differentially associated with these systems, such as “Do you experience heavy menstrual cycles?” (which would reinforce iron metabolism) or “Have you had recent thyroid laboratory testing?” (which would provide objective data for thyroid function assessment).

By way of example, the sequential prompting mechanism implemented by the user interface 111 improves the efficiency and user experience of the system 100 by avoiding unnecessary questions. For example, rather than presenting the user with an exhaustive questionnaire covering all possible symptoms and biomarkers, the user interface 111 dynamically generates targeted follow-up questions based on the current state of the staged reduction process. This approach reduces user burden, decreases the time required to complete the assessment, and improves the quality of collected data by focusing on the most diagnostically relevant signals.

By way of example, the user interface 111 may limit the maximum number of sequential prompts to a predetermined value, such as 10 questions, to prevent excessive user burden. Once the maximum number of prompts is reached, or once the threshold-based selection module 140 identifies a primary physiological system with sufficient confidence, the sequential prompting process terminates and the output generation module 150 generates the final recommendations.

### Working Example: Iron Metabolism Identification

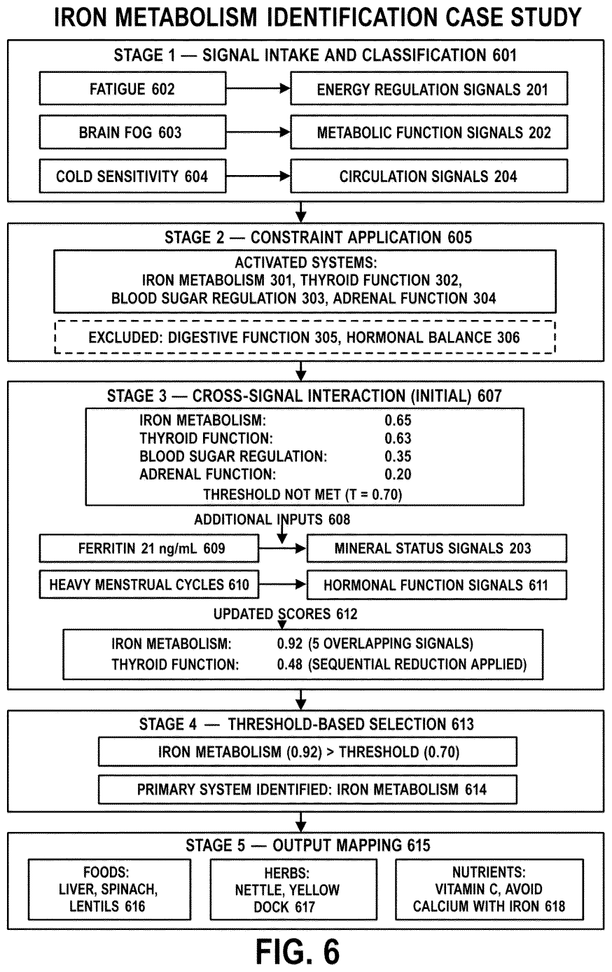

As illustrated in FIG. 6, the following example illustrates the operation of the system 100 in identifying iron metabolism 301 as the primary physiological system based on a sequence of user-reported health signals. This example is illustrated in FIG. 6 as a flowchart showing the stages of the staged constraint-based reduction process of the present invention.

Stage 1—Signal Intake and Classification 601: A user enters the following initial health signals through a signal input interface 111: fatigue 602, brain fog 603 and cold sensitivity 604. The signal classification module 110 queries the classification database 112 and categorizes these signals as follows: fatigue is categorized as energy regulation signals 201, brain fog is categorized as metabolic function signals 202 and cold sensitivity is categorized as circulation signals 204.

Stage 2—Constraint Application 605: For the categorized signal energy regulation signals 201, the constraint application module 120 queries the constraint database 121 and activates the following candidate physiological systems: iron metabolism 301, thyroid function 302, blood sugar regulation 303, and adrenal function 304. For the categorized signal metabolic function signals 202, the constraint application module 120 activates: iron metabolism 301, thyroid function 302, and blood sugar regulation 303. For the categorized signal circulation signals 204, the constraint application module 120 activates: iron metabolism 301 and thyroid function 302. At this stage, the activated candidate physiological systems are: iron metabolism 301 (activated by all three signals), thyroid function 302 (activated by all three signals), blood sugar regulation 303 (activated by two signals), and adrenal function 304 (activated by one signal). Systems outside this subset, such as digestive function 305 and hormonal balance 306 systems, are excluded from further evaluation.

Stage 3—Cross-Signal Interaction (Initial) 607: The cross-signal interaction module 130 calculates initial confidence scores for the activated candidate systems. The overlap detection component 131 identifies that iron metabolism 301 and thyroid function 302 are both activated by all three categorized signals, resulting in strong overlap support. The confidence level engine 133 calculates initial confidence scores as follows: iron metabolism 301=0.65 (three overlapping signals with moderate reliability weights), thyroid function 302=0.63 (three overlapping signals with moderate reliability weights), blood sugar regulation 303 =0.35 (two overlapping signals), adrenal function 304=0.20 (one signal). At this stage, no candidate system exceeds the predetermined threshold of 0.7 (for three categorized signals), so the threshold-based selection module 140 does not yet identify a primary physiological system.

Sequential Prompting: The user interface 111 determines that additional signals are needed to distinguish between iron metabolism 301 and thyroid function 302. The user interface 111 prompts the user with targeted follow-up questions: “Have you had recent blood work showing ferritin levels?” and “Do you experience heavy menstrual cycles?” The user responds with two additional health signals 608: ferritin level of 21 ng/mL 609 (below the normal range of 30-200 ng/mL for adult women) and heavy menstrual cycles lasting 7 days 610.

Stage 1 (Continued)—Signal Intake and Classification: The signal classification module 110 categorizes the new signals as follows: ferritin level of 21 ng/mL is categorized as mineral status signals 203 with high reliability weight (1.0 for laboratory biomarker), and heavy menstrual cycles lasting 7 days is categorized as hormonal function signals 611 with moderate reliability weight (0.7 for subjective report).

Stage 2 (Continued)—Constraint Application: For the categorized signal mineral status signals 302, the constraint application module 120 activates: iron metabolism 301 (strongly associated with ferritin levels). For the categorized signal hormonal function signals 611, the constraint application module 120 activates: iron metabolism 301 (heavy menstrual bleeding is a common cause of iron deficiency) and hormonal balance systems 306. The categorized signal mineral status signals 203 does not activate thyroid function 302, triggering the sequential reduction logic in the cross-signal interaction module 130.

Stage 3 (Continued)—Cross-Signal Interaction (Updated): The overlap detection component 131 identifies that iron metabolism 301 is now activated by five categorized signals (energy regulation 201, metabolic function 202, circulation 204, mineral status 203 and hormonal function 611), representing strong overlapping support. The conflict detection component 132 identifies that thyroid function 302 was previously activated by three signals but was not activated by the subsequently categorized mineral status signals 203 (ferritin level), triggering sequential reduction logic that decreases the confidence score for thyroid function 302. The confidence level engine 133 recalculates confidence scores as follows: iron metabolism 301=0.92 (five overlapping signals including high-reliability laboratory biomarker with weight 1.0), thyroid function 302=0.48 (reduced due to sequential reduction logic), blood sugar regulation 303=0.30, adrenal function 304=0.18, hormonal balance systems 306=0.35.

Stage 4—Threshold-Based Selection 613: The threshold comparison logic 141 compares the confidence scores to the predetermined threshold. With five categorized signals, the adaptive threshold mechanism adjusts the predetermined threshold to 0.7 (as illustrated in FIG. 4). Iron metabolism 301 with a confidence score of 0.92 exceeds this threshold, while all other candidate systems fall below the threshold. The primary system identifier 142 identifies iron metabolism 301 as the primary physiological system.

Stage 5—Output Mapping 615: The output generation module 150 retrieves the set of interventions associated with iron metabolism from the recommendation database 151. The intervention mapping logic 152 generates the following recommendations: (1) Food-based interventions 615: Increase consumption of iron-rich foods including red meat (beef, lamb), organ meats (liver), dark leafy greens (spinach, kale), legumes (lentils, chickpeas), and fortified cereals. Consume iron-rich foods with vitamin C sources (citrus fruits, bell peppers, tomatoes) to enhance absorption. (2) Herbal interventions 617: Nettle leaf (Urtica dioica) tea or tincture, 2-3 cups daily or 2-4 mL tincture three times daily; Yellow dock root (Rumex crispus) tincture, 2-4 mL three times daily. (3) Lifestyle interventions: Avoid consuming tea, coffee, or calcium supplements within 2 hours of iron-rich meals, as these inhibit iron absorption. Consider iron supplementation under medical supervision if dietary interventions are insufficient (ferrous sulfate 325 mg once or twice daily, or ferrous gluconate for better tolerability). Address underlying cause of iron loss: consult healthcare provider regarding heavy menstrual cycles; consider hormonal contraceptives or other treatments to reduce menstrual blood loss. (4) Specific nutritional support 618: Vitamin C supplementation (500-1000 mg daily) with iron-rich meals to enhance absorption; Avoid calcium and magnesium supplements concurrent with iron intake; Consider vitamin B12 and folate supplementation to support red blood cell production.

Those of ordinary skill in the art will recognize that the recommendations generated in this example are system-level recommendations targeting iron metabolism 301, rather than symptom-level recommendations that would address individual symptoms such as fatigue or brain fog in isolation. By identifying iron metabolism 301 as the underlying physiological system responsible for the constellation of symptoms (fatigue, brain fog, cold sensitivity) and biomarker findings (low ferritin, heavy menstrual cycles), the system 100 provides comprehensive interventions that address the root cause of the user's health concerns.

### Additional Examples

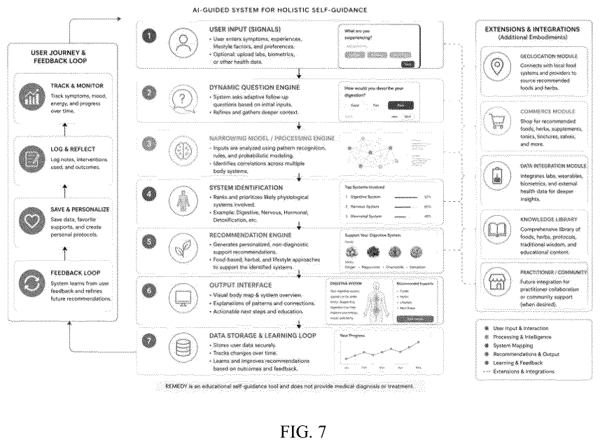

FIG. 7 illustrates a staged constraint-based physiological recommendation system according to an exemplary embodiment of the present invention.

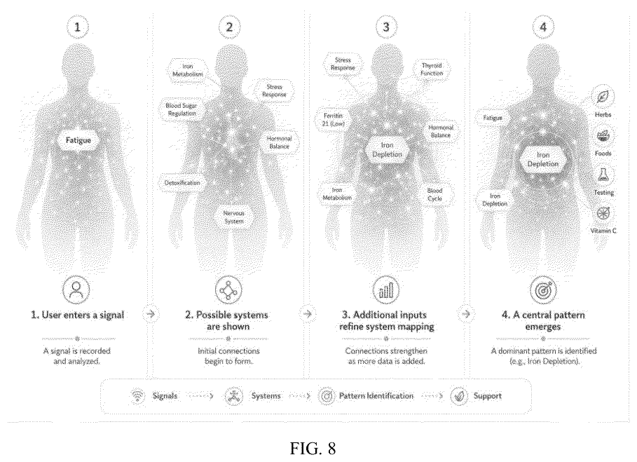

FIG. 8 illustrates a staged constraint-based physiological recommendation system according to an exemplary embodiment of the present invention.

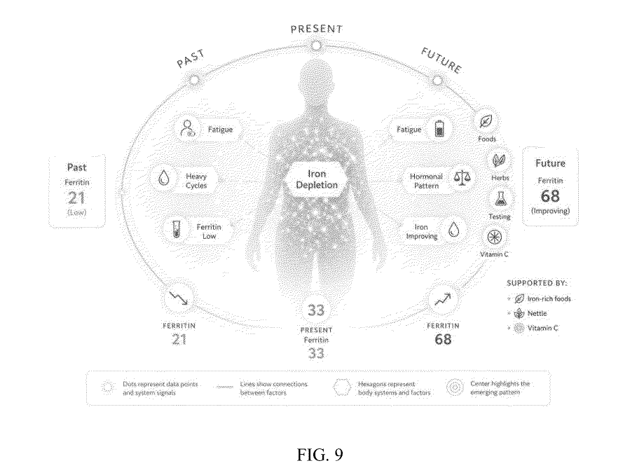

FIG. 9 illustrates a longitudinal system mapping interface showing temporal progression of user inputs, system relationships, and identified patterns according to an exemplary embodiment of the present invention.

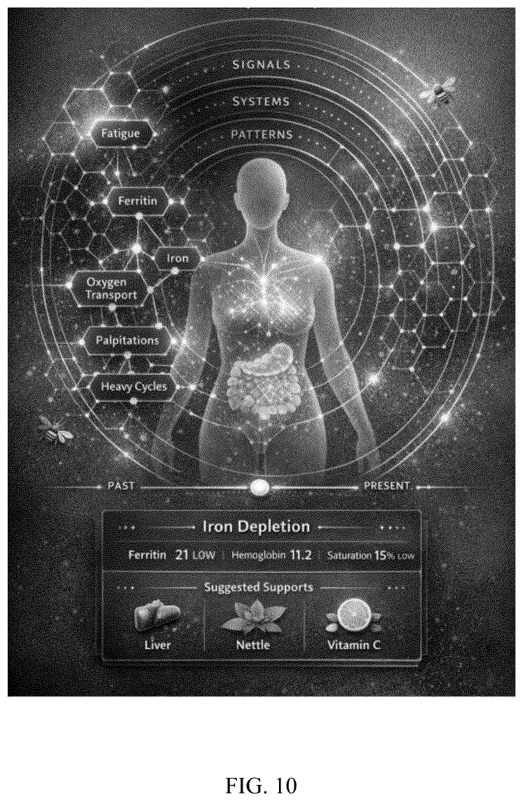

FIG. 10 illustrates an output interface of the system according to an exemplary embodiment of the present invention.

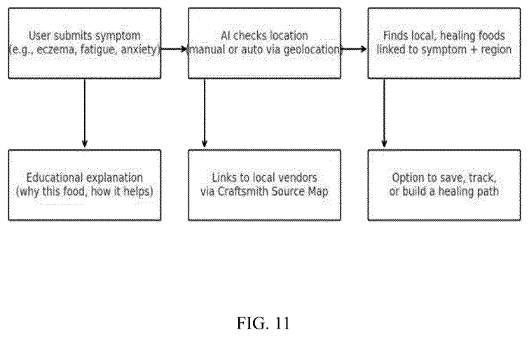

FIG. 11 illustrates an exemplary embodiment of a system workflow of the present invention incorporating geolocation-based recommendation functionality. In an exemplary embodiment, the invention may include a geolocation recognition component. For example, in one example workflow, the methods and systems 100 of the present invention may determine a user's region (e.g., State, Zip Code, etc.) or allow for manual selection/input of region by the user, for example to ensure that food recommendations are locally sourced or seasonal. In this regard, the methods and systems 100 of the present invention may comprise enabling longitudinal tracking of user inputs and outcomes. In this regard, the methods and systems 100 of the present invention may comprise identifying geographically relevant sources for recommended food or herbal interventions. By way of example, the methods and systems 100 of the present invention may also comprise a holistic food database, which database may combine ancestral/functional food wisdom with scientific data to assist in determining the healing properties or other health benefits for each food. By way of example, foods may be tagged with healing and/or health properties (e.g., gut-healing, anti-inflammatory, mineral-rich, etc.).

### Technical Advantages and Effects

Those of ordinary skill in the art will recognize that the staged constraint-based reduction process implemented by the system 100 is able to achieve significant technical advantages over prior health recommendation systems. First, the system 100 is able to achieve reduced computational complexity by progressively eliminating incompatible physiological systems at each stage rather than evaluating all possible system combinations simultaneously.

Second, the system 100 is able to achieve improved diagnostic precision through the cross-signal reinforcement and conflict logic implemented by the cross-signal interaction module 130.

Third, the system 100 is able to achieve enhanced accessibility by operating without requiring real-time biosensor data. The system 100 is able to accept user-reported symptoms, lifestyle factors, dietary habits, and optional laboratory biomarker values obtained from periodic testing, without requiring continuous biosensor monitoring. This enables the system 100 to serve users who do not have access to wearable biosensor devices, implantable sensors, or continuous monitoring infrastructure.

Fourth, the system 100 is able to achieve reduced memory 102 usage through the staged constraint-based reduction process. By excluding incompatible physiological systems at each stage, the system 100 maintains a smaller working set of candidate systems in memory 102 during the evaluation process.

Fifth, the adaptive threshold mechanism implemented by the threshold-based selection module 140 achieves improved balance between sensitivity and specificity. By dynamically adjusting the predetermined threshold based on the total number of categorized signals received, the system 100 avoids premature system identification when insufficient evidence is available (high threshold for few signals) while also avoiding overly stringent requirements when substantial evidence is available (lower threshold for many signals).

### Alternative Embodiments and Variations

In some exemplary embodiments, the system 100 may directly map categorized signals to recommendations without constraint-based reduction, though this approach may sacrifice the computational efficiency and diagnostic precision advantages of the staged process. In such exemplary embodiments, the signal classification module 110 categorizes user-reported health signals into predefined signal types, and the output generation module 150 retrieves recommendations associated with each signal type from the recommendation database 151, without performing constraint application, cross-signal interaction, or threshold-based selection. This simplified approach may be suitable for applications where computational resources are extremely limited or where rapid generation of preliminary recommendations is prioritized over diagnostic precision.

Alternatively, in other exemplary embodiments, the system 100 may perform constraint application and threshold-based selection without cross-signal interaction, relying solely on initial signal categorization to activate and exclude systems. In such embodiments, the constraint application module 120 activates a limited subset of candidate physiological systems for each categorized signal, and the threshold-based selection module 140 identifies the primary physiological system based on the number of signals that activate each system, without calculating confidence levels and/or scores that account for signal overlap and conflict. While simpler to implement, this approach does not achieve the reinforcement and conflict logic benefits provided by the cross-signal interaction module 130.

In another exemplary embodiment, cross-signal interaction may be performed on all physiological systems without prior constraint-based elimination, though this increases computational load. In such embodiments, the signal classification module 110 categorizes user-reported health signals, and the cross-signal interaction module 130 calculates confidence scores for all physiological systems in the plurality of physiological systems based on overlap and conflict relationships, without the constraint application module 120 excluding systems at each stage. The threshold-based selection module 140 then identifies the primary physiological system based on the confidence scores. This approach retains the diagnostic precision benefits of cross-signal interaction but sacrifices the computational efficiency benefits of staged constraint-based reduction.

Additional signal types beyond those explicitly described may include but are not limited to: immune function signals (e.g., frequent infections, slow wound healing), neurological signals (e.g., numbness, tingling, memory issues), cardiovascular signals (e.g., chest pain, palpitations, shortness of breath), respiratory signals (e.g., chronic cough, wheezing), musculoskeletal signals (e.g., joint pain, muscle weakness), and dermatological signals (e.g., rashes, hair loss, nail changes). Preferably, each additional signal type is associated with a corresponding subset of candidate physiological systems in the constraint database 121.

In addition to food-based, herbal, and lifestyle interventions, recommendations generated by the output generation module 150 may include but are not limited to: supplement recommendations (specific vitamins, minerals, amino acids, or other nutritional supplements), exercise protocols (specific types, frequencies, and intensities of physical activity), stress management techniques (meditation, breathing exercises, progressive muscle relaxation), sleep optimization strategies (sleep hygiene practices, circadian rhythm regulation), and environmental modifications (reducing exposure to toxins, allergens, or other environmental factors). The recommendation database 151 may be expanded to include these additional intervention types for each physiological system.

The foregoing description sets forth specific embodiments of the invention. It should be recognized, however, that such description is not intended as a limitation on the scope of the present disclosure. Various modifications, combinations, and alternatives to the exemplary embodiments described herein will be apparent to those skilled in the art. For example, the specific signal types, physiological systems, confidence scoring formulas, threshold values, and recommendation types described herein are illustrative and may be modified, expanded, or replaced with alternative implementations without departing from the core inventive concept of staged constraint-based reduction with cross-signal interaction for physiological system identification. The scope of the invention is defined by the appended claims rather than by the foregoing description, and all equivalents, variations, and modifications that fall within the scope of the claims are intended to be embraced therein.

It will be appreciated that the above description is merely exemplary in nature and is not intended to limit the present disclosure, its application or uses. Therefore, it is intended that the present disclosure not be limited to the particular examples illustrated by the drawings and described in the specification as the best mode presently contemplated for carrying out the teachings of the present disclosure, but that the scope of the present disclosure will include any embodiments falling within the foregoing description and the appended claims.

We claim:

1. A physiological recommendation system comprising:
a processor; and
a memory storing instructions that, when executed by the processor, cause the system to:
receive at least one user-reported health signal;
categorize the at least one health signal into at least one predefined signal type, the at least one signal type associated with a subset of physiological systems from a plurality of physiological systems;
for each categorized signal, activate a limited subset of candidate physiological systems and exclude physiological systems outside said subset from further evaluation;
perform cross-signal interaction by:
increasing a confidence level for a candidate physiological system when multiple categorized signals overlap in activating a candidate system, and
reducing the confidence level for the candidate physiological system when categorized signals conflict;
identifying a primary physiological system when the confidence level for the system exceeds a predetermined threshold across a minimum number of categorized signals; and
generating at least one recommendation based on at least one identified primary physiological system, wherein the at least one recommendation comprises at least one intervention.

2. The system according to claim 1, wherein the cross-signal interaction further comprises reducing the confidence level for the candidate physiological system when a subsequently categorized signal fails to activate the candidate system after the system was previously activated by an earlier categorized signal.

3. The system according to claim 1, wherein the at least one signal type is selected from the group comprising energy regulation signals, metabolic function signals, mineral status signals, and circulation signals; and each at least one signal type is associated with at least one physiological system.

4. The system according to claim 1, wherein the plurality of physiological systems is selected from the group comprising iron metabolism, thyroid function, blood sugar regulation, adrenal function, digestive function, and hormonal balance systems.

5. The system according to claim 1, wherein the confidence level is a confidence score calculated as a weighted sum of signal overlap factors, wherein each signal overlap factor is weighted based on a reliability metric associated with the signal type.

6. The system according to claim 1, wherein the predetermined threshold is dynamically adjusted based on a total number of categorized signals received, wherein the threshold decreases as the number of signals increases.

7. The system according to claim 1, wherein the user-reported health signals are selected from the group comprising subjective symptoms, lifestyle factors, dietary habits, and laboratory biomarker values.

8. The system according to claim 1, further comprising a user interface module configured to sequentially prompt the user for additional health signals based on the currently activated subset of candidate physiological systems.

9. The system according to claim 1, wherein the set of recommendations further comprises nutritional support recommendations selected from the group comprising specific food items, herbal supplements, vitamins and minerals, the selection based on the identified primary physiological system.

10. The system according to claim 1, wherein the at least one intervention is selected from the group comprising food-based interventions, herbal interventions and lifestyle interventions.

11. A computer-implemented method for generating physiological recommendations, the method comprising:
receiving, by a processor, at least one user-reported health signal;
categorizing, by the processor, the at least one health signal into at least one predefined signal type, the at least one signal type associated with a subset of physiological systems from a plurality of physiological systems;
for each categorized signal, activating a limited subset of candidate physiological systems and excluding physiological systems outside said subset from further evaluation;
performing cross-signal interaction by:
increasing a confidence level for a candidate physiological system when multiple categorized signals overlap in activating a candidate system, and
decreasing the confidence level for a candidate physiological system when categorized signals conflict;
identifying a primary physiological system when the confidence level for the system exceeds a predetermined threshold across a minimum number of categorized signals; and
generating at least one recommendation based on at least one identified primary physiological system, wherein the at least one recommendation comprises at least one intervention.

12. The method according to claim 11, wherein the categorizing step comprises mapping the at least one health signal to a predefined signal type using a classification database comprising signal-to-type mappings.

13. The method according to claim 11, wherein the activating step comprises retrieving, from a constraint database, a list of candidate physiological systems associated with the at least one predefined signal type, wherein the constraint database defines signal-to-system mappings.

14. The method according to claim 11, wherein the cross-signal interaction is performed iteratively as each at least one health signal is categorized, and updating the confidence level after each categorization.

15. The method according to claim 11, wherein the identifying step further comprises comparing the confidence levels of multiple candidate physiological systems that exceed the predetermined threshold and selecting the candidate system with the highest confidence level as the primary physiological system.

16. The method according to claim 11, wherein the generating step comprises retrieving, from a recommendation database, the at least one intervention associated with the identified primary physiological system, wherein the recommendation database maps each physiological system to corresponding food-based, herbal and lifestyle interventions.

17. The method according to claim 11, further comprising presenting the at least one recommendation to the user via a user interface, wherein the at least one presentation includes an explanation of the identified primary physiological system and rationale for each recommendation.

18. The system according to claim 11, wherein the minimum number of categorized signals required for identifying the primary physiological system is at least three signals.

19. The system according to claim 11, wherein the system is configured to identify a secondary physiological system when the confidence level for a second candidate system exceeds a secondary threshold and to generate at least one supplemental recommendation.

20. The system according to claim 11 wherein the at least one intervention is selected from the group comprising food-based interventions, herbal interventions and lifestyle interventions.

21. A non-transitory computer-readable storage medium storing instructions that, when executed by a processor, cause the processor to perform the method according to claim 11.
